(19) United States
(12) Patent Application Publication
(10) Pub. No.: US 2004/0176467 A1
(43) Pub. Date: Sep. 9, 2004

(54) Method of preventing or reducing the occurrence of symptoms of psychosis

(76) Inventor: Neil Richtand, Cincinnati, OH (US)

Correspondence Address: Dykema Gossett, Suite 300, 39577 Woodward Avenue, Bloomfield Hills, MI, 48304, US

(21) Appl. No.: 10/486,593

(22) PCT Filed: Aug. 9, 2001
(86) PCT No.: PCT/US01/24891

(51) Int. Cl. A61K 31/137

(52) U.S. Cl. 514/649

(57) ABSTRACT

A method of preventing or suppressing symptoms of psychosis by treating non-psychotic patients who are at risk of developing psychosis. The method includes determining whether a patient is at risk for developing psychosis; making a diagnosis that the patient is at risk; and administering to the patient a selective D3 antagonist prior to the time the patient is psychotic in an amount sufficient to prevent or suppress symptoms of psychosis.

### FIELD OF THE INVENTION

The present invention deals generally with the treatment of psychosis and, more specifically, with methods of preventing or reducing the occurrence of the symptoms of psychosis in humans.

### BACKGROUND OF THE INVENTION

A number of therapeutic agents are available for use in the treatment of psychosis. For example, neuroleptics, such as phenothiazines, and similar compounds which are pharmacologically active on the dopamine receptor system, provide significant benefits to patients diagnosed with psychotic disorders such as schizophrenia.

Anti-psychotic agents have traditionally been administered following significant manifestations of psychotic symptoms, i.e., after the patient has become psychotic, a disruptive event for the patient and others.

As will be understood by those skilled in the art, however, the development of psychosis is generally preceded by a prodromal phase “Initial prodrome” in psychotic illnesses refers to the early symptoms that precede the first occurrence of psychosis in an individual. That is, prior to the first onset of psychosis, a patient typically develops behavioral symptoms which do not rise to the level of psychotic symptoms, but which denote a pre-psychotic period.

Prodromal symptoms which have been identified for first-episode psychotic illnesses include: a reduction in the ability to concentrate; a reduction in personal motivation; depressed mood; sleep disturbances; anxiety; social withdrawal; suspiciousness; deterioration in role functioning; and irritability. The interval between the onset of prodromal symptoms to the onset of psychotic symptoms varies, but it is generally agreed that most patients who develop psychosis experience prodromal symptoms before the first episode of psychosis.

In addition to prodromal symptoms in psychotic illnesses, there are also predictive risk factors such as a family history of psychosis. For example, evidence exists that the biological offspring of parents, one or both of whom have been diagnosed as suffering from psychosis, are believed to be at greater risk for developing a psychotic illness. Some studies have found an even greater risk when an offspring's mother suffers from psychosis, than when only the father has manifested psychosis.

Other predictive risk factors for psychotic illness include environmental factors, such as exposure to extreme stress. For example, it has been reported that the risk of developing a psychotic illness while exposed to the medical or surgical stress of the intensive care unit exceeds 40%.

Accordingly, it would be desirable to provide a treatment for psychosis which would prevent, avoid or suppress the development of the illness, rather than simply treat the symptoms after the patient has developed the illness.

### SUMMARY OF THE INVENTION

The present invention provides a method for the prevention or suppression of symptoms of psychosis. The method includes the steps of evaluating a human patient for factors associated with the risk of developing psychosis, which in one aspect are non-symptomatic factors such as a family history of psychosis or biological markers indicative of a high risk of developing psychosis or identification of high-risk factors, such as presence in an intensive care unit, and which in another aspect are prodromal symptoms associated with the prodromal phase of psychosis; making a diagnosis based on this evaluation that the patient is either at-risk (i.e., at greater risk than the general population) of developing a psychosis, or that the patient is in the prodromal phase of a psychotic illness; and administering a selective D3 antagonist to the patient in an amount and on a schedule which is efficacious in preventing the expression of symptoms indicative of a psychotic illness.

In one aspect, the D3 antagonist is either nafadotride, certain naphthamide derivatives which act as selective D3 antagonists, NGB 2849, U99194A, (+)-AJ 76, (+)-UH 232, PD 152255, NGB 2904, S33084, GR103,691, GR218,231, (+)-S14297, or SB-277011.

### DETAILED DESCRIPTION OF THE PREFERRED EMBODIMENT

Dopamine is a catecholamine neurotransmitter found in neurons of both the central and peripheral nervous systems. It is stored in vesicles in axon terminals and released when the neuron is depolarised. Dopamine interacts with specific membrane receptors to produce its effects. These effects are terminated by re-uptake into the presynaptic neuron by a dopamine transporter, or by metabolic inactivation by monoamine oxidase B (MAO-B) or catechol-0-methyltransferase.

A dopamine receptor regulatory pathway that plays a crucial role in rat locomotor sensitization has been implicated in the development of human psychosis. As explained more fully hereinafter, differential binding of dopamine to the D3 dopamine receptor, in comparison to the D1/D2 receptors, initiates a homoeostatic reaction in rats that ultimately causes sensitization.

The binding of dopamine to D3 receptors results in less locomotor activity, while the binding of dopamine to D1/D2 receptors results in greater locomotor activity in rodents. Drugs such as cocaine and amphetamines work to prolong the presence of dopamine within neuronal synapses. Because D3 receptors have a greater affinity for dopamine than D1 or D2 receptors, the greater amount of dopamine in the synapses will result in D3 receptors being more highly bound or saturated with dopamine. This theoretically should decrease the rat's locomotor activity, as D3 mechanisms are inhibitory. Additionally, the animal body's natural reaction to the inhibition is to attempt to return to normal conditions, thus over a period of time recruiting mechanisms to increase locomotor activity at the next exposure to cocaine or amphetamine.

Over time, organisms learn to adapt to the excess presence of dopamine on its D3 receptors by counteracting the inhibitory effects. Thus, once the D3 receptors have been over-occupied, “homeostatic” mechanisms are recruited which compensate for what the body senses is the impending inhibitory effect. This results in an increased effect of smaller dosages of drugs (e.g.,cocaine) over a prolonged period of drug-use (i.e., sensitization). This is the opposite of tolerance, which is the phenomenon of requiring greater dosages of drugs over time to achieve the same effect on the body. Thus, in a rat that, through repeated drug use has been sensitized to amphetamine, a smaller dose of the drug is effective in creating a similar effect as an earlier, higher dose.

In accordance with the present invention, the homoeostatic responses to increased D3 receptor binding of dopamine is a key component in the genesis of sensitization of rat locomotion and in the development of human psychosis. In accordance with the present invention, a D3 antagonist preventing excess dopamine binding at the D3 receptor, by stopping sensitization, prevents the onset of symptoms of such illnesses as schizophrenia, schizoaffective disorder, bipolar affective disorder, and delirium if administered in the prodromal phases of the disease or to high-risk, asymptomatic individuals.

The five dopamine receptor subtypes (D1-D5) are members of the superfamily of G protein-coupled receptors. Dopamine receptors have been known since 1978 to be divided between two distinct families differing in biochemical and pharmacological properties. In vitro, D1-family receptors (D1 and D5) couple to Gs stimulatory proteins, activating adenylyl cyclase, while D2-family receptors (D2, D3, D4) couple to Gi inhibitory proteins, inhibiting adenylyl cyclase. The G protein and second messenger systems affected by dopamine receptors in vivo, however, have not been clearly established. Dopamine receptors couple effectively to a wide range of signaling cascades in vitro, including calcium channels, phospholipase C, potassium channels, arachidonic acid release, Na+/H+ exchangers, Na+-H+-ATPase, and cell growth and differentiation pathways. These observations suggest that dopamine mediates a complex array of neural signaling pathways in vivo.

Cloning of the D2 dopamine receptor led to the subsequent cloning and characterization of the other dopamine receptor subtypes. Although the D3 dopamine receptor has been cloned and extensively characterized, the second messenger signaling pathways affected in brain through the D3 receptor are not known. Recombinant D3 receptors expressed in cell culture couple to a variety of signaling cascades, dependent on host cell, including both stimulation and inhibition of adenylyl cyclase, change in K⁺ and Ca⁺² current, mitogenesis, and increased extracellular acidification. The effect of D3 receptor occupation on cell firing rate has not been clearly determined. Although it is known that the preferential D3 receptor agonist 7-hydroxy-N,N-di-n-propyl-2-aminotetralin (7-OH-DPAT) inhibits neuronal firing rate in substantia nigra (SN), ventral tegmentum (VTA), and nucleus accumbens (NA), suggesting D3 receptor occupation may inhibit neuronal firing, this same effect of D3 agonist was also observed by others in SN and VTA of D3 knockout mice, indicating the observed neuronal inhibition may be mediated through D2 receptor activation.

It is known that D3 dopamine receptor mRNA and protein expression are limited primarily to olfactory tubercle, nucleus accumbens, and islands of Calleja (located ventral to the ventral pallidum and nucleus accumbens), phylogenetically ancient limbic brain regions linked to motivated and emotional behaviors. The earliest reports describing the highly restricted expression pattern of the D3 receptor suggested a role for this receptor in psychosis. It is also known that protein and mRNA expression are highly co-localized, suggesting receptor expression occurs primarily on perikarya, proximal dendrites, and short axons as opposed to long axon terminals from other brain regions. The cellular pattern of D3 protein expression does not overlap with expression of synaptic proteins such as synaptophysin, suggesting that receptor localization is primarily extrasynaptic.

Co-localization studies by others of D1, D2 and D3 receptors indicate that the majority of D3 expressing neurons in islands of Calleja and nucleus accumbens shell also express D1 receptor mRNA. And, it is known that in human brain, most D3 mRNA expressing cells also express D2 mRNA, while in rodent brain, in contrast, D2 and D3 receptors appear to have predominantly complementary rather than overlapping patterns of expression.

The physiological consequence of dopamine receptor subtype co-expression is variable, leading in some cases to synergistic effects and in other cases to antagonistic effects, perhaps dependent upon the second messenger systems expressed within a given neuron. Several findings by those skilled in the art have demonstrated a complex functional interrelationship between D1, D2, and D3 receptors at the cellular level.

Activation of DA D3 and D2 receptor subtypes has, in some if not all cases, opposing functional consequences on behavior. This has been most clearly characterized with regard to rodent locomotion, which is regulated by the opposing balance of D3 and D1/D2 receptor activity. Considerable evidence supports the widely held view that D1/D2 activation increases locomotion, while D3 receptor stimulation inhibits locomotion.

Mice with targeted deletion of the D3 DA receptor gene and wild-type mice show similar locomotion during both light and dark cycles. In contrast, others have demonstrated that D3 DA receptor knock-out mice exhibit increased locomotion in response to a novel environment or following treatment with low dose cocaine and amphetamine, presumably due to lack of an inhibitory response opposing the action of D1 and D2 receptor activation. In a similar fashion, it is known that rats infused intracerebroventricularly with antisense oligonucleotide to the D3 dopamine receptor also display increased locomotion in response to a novel environment, in addition to decreased high affinity spiperone binding in limbic forebrain consistent with decreased D3 dopamine receptor expression. A separate study by others found increased locomotion response to the mixed dopamine receptor agonist apomorphine in rats treated intracerebroventricularly with antisense oligonucleotide to the D3 dopamine receptor, again suggestive of an inhibitory role on locomotor activity for the D3 receptor.

In a fashion similar to the opposing roles of D2 and D3 receptors in locomotion, evidence suggests opposite roles for D2 and D3 receptors in memory consolidation, with DA D2 receptors facilitating and D3 receptors inhibiting memory consolidation.

Pharmacological studies by others with D3 preferring agonists such as 7-OH-DPAT, and PD 128907 also suggest the D3 receptor is inhibitory to rodent locomotion. These studies generally report a biphasic effect on rat locomotion, with inhibition of spontaneous locomotion at low doses devoid of significant D2 receptor occupancy, and stimulation of locomotion at higher doses, likely due to D2 receptor activation. It is known that a similar inhibition of locomotion is observed following 7-OH-DPAT micro-injection into rat nucleus accumbens, and following systemic administration in mice. However, studies in two different laboratories, using mice with deletions of the gene encoding the D3 dopamine receptor, found that dopamine receptor agonist doses previously thought to exert behavioral effects through the D3 receptor may also exert behavioral effects through D2 receptor activation. Neither laboratory found a significant difference in the hypolocomotor behavioral response to PD 128,907 or 7-OH-DPAT between D3 knockout and wild type mice, whereas the hypomotoric effects were abolished in mutant mice lacking the D2 receptor. Although all but one of these studies employed 7-OH-DPAT doses at least 10-fold higher than the 10 ug/kg dose inhibitory to rodent locomotion in other investigations, these results were interpreted by both laboratories as suggesting that some of the locomotor inhibitory effects of 7-OH-DPAT previously thought to result from D3 activation may also be mediated through D2 or other receptors.

The earliest D3 receptor knockout study reporting the hypolocomotor effect of D3 receptor activation, as well as antisense oligonucleotide studies reaching a similar conclusion, evaluated D3 modulation of exploratory behavior in non-habituated animals. In contrast, the more recent studies that failed to observe a D3-mediated hypolocomotor effect of D3 agonists examined locomotion in habituated animals. There is considerable evidence that unique mechanisms modulate novelty-induced exploratory locomotion. Taken together, these data suggest the divergent findings evaluated D3 modulation of different forms of locomotion, and that D3 receptor activation inhibits novelty-induced exploratory locomotor behavior.

It is known that D3 preferring antagonists generally produce dose-dependent effects opposite to those of D3 agonists, in some but not all studies. It has been shown that the D3 antagonists U99194A, nafadotride, (+)-AJ 76, (+)-UH 232, and PD 152255 all stimulate rat locomotion. The D3 antagonists generally exhibit a biphasic response, increasing locomotion at low doses and inhibiting locomotion at higher doses. Because D3 antagonist doses stimulating locomotion have low D2 receptor occupancy, while higher doses inhibitory to locomotion significantly occupy D2 receptors, the locomotor stimulant effect is believed due to D3 receptor blockade, while the locomotor inhibitory effect is believed secondary to blockade of D2 receptors. It is known that low doses of D3 antagonists also augment the locomotion response to amphetamine and cocaine, and inhibit stimulant-induced locomotion at higher doses. Nafadotride has exhibited a 10-fold preference for D3/D2 . In addition, several compounds have recently been synthesized by others with significantly greater D3 receptor selectivity, such as NGB 2849 (291-fold preference for D3/D2), NGB 2904 (155-fold preference for D3/D2), and S33084 (100-fold selectivity for D3/D2). Taken together, the available data support the prevailing model that the locomotion response to D3 DA receptor stimulation is opposite to the effect of concurrent D1 and D2 receptor stimulation at the integrative systems level.

It has been proposed by others that transient down regulation of the D2 DA autoreceptor's locomotor inhibitory influence contributed to development of sensitization. Early work hypothesized that D3 agonists might also inhibit locomotion through a pre-synaptic autoreceptor mechanism, inhibiting the firing of dopaminergic cell bodies and dopamine synthesis and release at nerve terminals. Some investigators have detected very low expression of D3 mRNA restricted to a portion of tyrosine hydroxylase positive neurons in the lateral substantia nigra and ventral tegmentum. D3 receptor immunoreactivity has also been identified by others in both substantia nigra and ventral tegmentum. Studies comparing wild-type and dopamine receptor knockout mice demonstrate a loss of autoreceptor inhibition of DA release in synaptosomes of D2 receptor knockout mice, while wild-type and D3 receptor knock out mice have similar autoreceptor function. Taken together, these data demonstrate that a significant role for the D3 receptor in autoreceptor regulation of DA release is unlikely.

The expected physiological response to repetitive drug administration is tolerance. That is, drug effects generally become smaller with repeated usage, requiring more and more drug to achieve the same endpoint. Although numerous factors are thought to contribute to the development of tolerance, a compelling case can be made that learning (conditioning) is important. Drugs cause changes in critical parameters that are regulated by the body (e.g., body temperature; or the background tone over motor control systems). When drug-induced changes are detected, they trigger reflexes that bring the parameter back toward the pre-drug level. This is the fundamental principle of homeostasis.

Considered in this light, sensitization appears to be a quite different phenomenon from tolerance, one that is in fact maladaptive. That is, because stimulant drugs increase motor activity, the tolerance model predicts that an individual would learn to reduce motor activity and thereby circumvent drug-induced disruptions of its motor control system. For this reason, the D3 dopamine receptor behavioral action as a “brake” on D1/D2 mediated behaviors makes the D3 receptor an attractive candidate for tolerance through dopamine signaling pathways contributing to sensitization. While not wishing to be bound by theory, in the present invention behavioral sensitization is a result of multiple neural controls over a behavior, more than one of which is impacted by the same drug. For example, dopamine influences locomotion in multiple ways by interacting with D1, D2, and D3 dopamine receptors. It is further believed that when dopamine levels are increased following pharmacologic challenge, tolerance develops differentially for each of these three receptor systems. Because the receptor subtypes differ widely in affinity for dopamine, this results in a relatively greater tolerance to D3 DA receptor signaling than to D1 or D2 signaling, and therefore an imbalance that favors the activity of D1 and D2 receptors over the D3 receptor. Thus, rather than reflecting a true augmentation of a drug effect, behavioral sensitization is a manifestation of the same phenomenon that underlies tolerance; i.e., a decrease of a drug effect.

The D3 dopamine receptor has the highest affinity for dopamine among the dopamine receptors, which differ widely in affinity for neurotransmitter. Dopamine receptors are believed to exist in at least two affinity states, a high affinity and a low affinity state. In studies by others, when binding affinity to D2 receptors was determined in intact, viable anterior pituitary cells by both direct and indirect methods, no high-affinity binding was found, suggesting that endogenous guanine nucleotides effectively eliminate the high-affinity binding state in vivo. The high-affinity state is predominantly responsible for D2 dopamine receptor activity, however, suggesting that the low-affinity state may be the predominant affinity state in vivo, while a transient, small proportion of receptors in high-affinity state account for most receptor activity.

It is known that dopamine receptor subtypes have different affinities. Measurements of dopamine concentration in the central nervous system suggest the difference in affinity between dopamine receptor subtypes has important physiological significance. Indirect estimation by others of synaptic dopamine concentration by competition kinetics between radiolabeled dopamine antagonists and endogenous dopamine suggests an average synaptic dopamine concentration of approximately 50 nM. The confined synaptic space does not allow direct dopamine concentration measurement at dopamine receptors, although direct measurement of dopamine extracellular fluid concentration adjacent to the synaptic space has been performed by a variety of methods with similar results. Intracerebral microdialysis studies by others generally report basal dopamine extracellular levels between 3 to 5 nM. And, it is known that direct electrochemical measurement of extracellular fluid dopamine concentration adjacent to the synaptic space by fast-scan cyclic voltammetry, which provides temporal and spatial resolution not achievable by intracerebral microdialysis, indicates unstimulated synaptic dopamine concentration of less than 6 nM. Measurements by others of transient stimulated extracellular dopamine concentrations range from 120 nM during high frequency stimulation trains to approximately 250 nM following a single-stimulus pulse, suggesting transient synaptic dopamine concentrations as high as 1.6 mM, taking into account the geometry of the synaptic cleft. Following stimulation, the dopamine concentration drops rapidly, with clearance by diffusion and removal by the dopamine transporter. When the dopamine transporter is blocked by stimulant drugs such as cocaine or amphetamine, clearance is slowed, and the decline in dopamine concentration is slowed. This leads to prolonged periods of elevated dopamine concentrations, with average concentrations in the range of 750 nM as determined by microdialysis. The 70-fold greater affinity for dopamine dictates low-affinity state D3 occupancy of 96% at this dopamine concentration, compared to occupancy of 25% for D1 and 27% for D2 receptors. These marked differences in receptor occupancy could result in greater signaling through D3 relative to D1 or D2 receptors, if other factors affecting receptor function such as receptor reserve and coupling efficiency were relatively equivalent. Since the expected physiological response to stimulant-induced dopamine release is a homeostatic response to return to equilibrium, the homeostatic response of D3 signaling pathways to abnormally elevated (and perhaps continuous) occupancy will be greater than the homeostatic responses through D1 and D2 signaling pathways. This would lead to greater decrease in signaling through D3 pathways following repetitive amphetamine exposure, relative to the decreased response through D1 and D2 pathways, and therefore a decrease in the “brake” on D1/D2 mediated response to the next amphetamine exposure. Thus, in the present invention, it is believed that an imbalance between homeostatic responses to the D3 versus D1 and D2 signaling pathways contributes to behavioral sensitization to stimulant drugs.

Behavioral sensitization results in part from properties of the “motive circuit” which mediates sensitization. Neuronal activity within the nucleus accumbens is regulated by both glutamatergic and dopaminergic systems in an interdependent fashion, and accumbens DA release is also reciprocally regulated by DA activity in other brain regions including prefrontal cortex (PFC) and amygdala. Expression of sensitization may involve an increase in stimulant-induced elevations of glutamate and dopamine within NA, and may therefore be critically dependent upon inputs from other brain regions. These observations suggest several potential mechanisms through which D3 receptor downregulation might contribute to enhanced responsiveness to stimulant drugs. It is known that excitatory amino acid (EAA) projections from PFC to NA and VTA are inhibited by D2-family DA receptors in PFC, and that down-regulation of this inhibitory tone stimulates accumbens DA release. If down regulation of D3 receptor function in the PFC increased dopaminergic function in nucleus accumbens, this could then lead to down regulation of D3 receptors in the nucleus accumbens. In a similar fashion, DA release in VTA activates D1 DA receptors facilitating glutamate release from projections from PFC. The resulting activation of dopaminergic projections from VTA to NA may then result in a prolonged elevation of DA in NA, again leading to down regulation of accumbens D3 receptors.

Such a cascade model explains how events in cortical and midbrain regions during the process of initiating sensitization would then trigger postsynaptic changes in dopaminergic and glutamatergic nucleus accumbens responsiveness thought important to expression of sensitization, and would account for the observed participation of both glutamatergic and dopaminergic systems in sensitization.

The DA D2-family “heteroreceptor” inhibiting glutamate release on glutamate nerve terminals provides a potentially separate mechanism through which D3 receptor down-regulation may contribute to increased glutamatergic responsiveness to stimulant drugs. Studies in several labs demonstrate that D2/D3 family receptors regulate glutamate release in caudate, NA and VTA through the action of a dopamine “heteroreceptor” on glutamate nerve terminals inhibiting glutamate release. While the efficacy of D2/D3 agonists such as pramipexole as neuroprotective agents is consistent with a D3 heteroreceptor inhibiting glutamate release, the dopamine receptor subtype of this glutamate-inhibiting receptor has not been definitively established.

The “D3 receptor mechanism” may explain aspects of the sensitization literature which have been difficult to reconcile. Numerous studies have shown that AMPH applied directly into NA does not elicit sensitization, while both cocaine administration and kindling in the NA does result in sensitization. This seemingly contradictory finding is accounted for by sensitization requiring differential saturation of D3 vs D1 and D2 receptors. AMPH releases DA to a greater extent than cocaine, because AMPH releases non-vesicular stores of cytosolic DA in addition to blocking DA re-uptake. Brief, extremely high local concentrations of AMPH achieved with direct drug infusion would be expected to result in extremely high local DA concentrations, saturating D1 and D2, as well as D3 receptors. Since all three receptor types would be fully saturated, this would not cause differential tolerance and would not result in sensitization, according to the present D3 receptor mechanism. In contrast, since cocaine blocks reuptake but does not release cytosolic DA, direct infusion of cocaine into NA would be expected to elevate DA concentration to a lesser degree than local AMPH infusion. This could provide for differential tolerance of D3 vs. D1 and D2 receptors, resulting in sensitization according to the present invention.

Other factors may also contribute to the inability of direct AMPH infusion into NA to elicit sensitization. It is known that repetitive stimulations are most effective in eliciting tolerance responses. The brief, transient receptor saturation occurring with local AMPH infusion is not impulse-dependent and may deplete local DA stores, resulting in a limited number rather than repetitive waves of receptor saturation and desaturation. In contrast, local cocaine infusion is dependent upon neural activity to increase extracellular dopamine concentration, and may lead to repetitive increases and decreases in receptor saturation with nerve stimulation. This contrast in temporal characteristics is believed to be another important variable contributing to the lack of sensitization elicited by direct AMPH application to NA. The observation by others that D1 dopamine receptor knockout mice exhibit locomotor sensitization suggests the likelihood that mechanisms other than ventral tegmental D1 dopamine receptor activation contribute to behavioral sensitization.

Evidence suggests manipulations exerting enduring changes in dopamine mediated behaviors also down-regulate DA D3 receptor in nucleus accumbens, suggesting that decreased D3 receptor function may mediate behavioral plasticity of DA systems from diverse causes. It is known that exposure of pregnant rats to stress in late pregnancy results in offspring with increased locomotor response to novelty, amphetamine self-administration, and more rapid sensitization of locomotor response. This manipulation also decreases D3 receptor, estimated by 7-OH-DPAT binding, in nucleus accumbens shell and core regions. Additionally, neonatal hippocampal lesion, a developmental model of hyper-dopaminergic behavior resulting in increased locomotion response to amphetamine, also leads to decreased D3 receptor binding in nucleus accumbens of lesioned animals.

Decreased D3 receptor function in prefrontal cortex may also contribute to sensitization. It is known that reduced inhibition of excitatory efferent projections from PFC may play a role in sensitization. Recent studies by others describe D2-family receptors in PFC inhibitory to stimulant-induced locomotion and stereotyped behavior. Following sensitization to cocaine or AMPH, they demonstrate loss of function of this D2-family receptor.

Studies employing non-selective dopamine receptor antagonists demonstrate a wide range of dissimilar behavioral effects. It is known that repetitive treatment with haloperidol (D2/D3 affinity ratio 5) results in sensitization to later challenge by cocaine, amphetamine, or the dopamine agonist apomorphine, and repetitive treatment with sulpiride (D2/D3 affinity ratio 2) results in sensitization to cocaine. In contrast, there have also been reports by others of blockade of sensitization with non-selective D2-family antagonists, and of D2-family antagonists without effect on sensitization.

Recent studies of more selective D3 DA receptor antagonists and agonists, however, indicate the potential for D3 receptor involvement in behavioral sensitization. The present inventors have found inhibition of locomotor sensitization to amphetamine by the D3 receptor antagonist nafadotride at a dose shown by in vivo receptor binding and behavioral studies to be devoid of appreciable D2 receptor occupancy. This finding is consistent with adaptive down-regulation of D3 dopamine receptor function contributing to the development of behavioral sensitization. Additionally, the present inventors have observed a decreased behavioral response to the D3 receptor agonist 7-OH DPAT following a sensitizing regimen of amphetamine. While the present inventors have not detected stable D3 DA receptor mRNA down-regulation in nucleus accumbens, striatum, or prefrontal cortex with a sensitizing treatment regimen of amphetamine, others have demonstrated that D3 receptor protein estimated by 7-OH-DPAT binding is significantly reduced in nucleus accumbens in response to cocaine treatment causing sensitization of the locomotion response. In contrast, however, other investigators report D3 receptor mRNA and 7-OH-DPAT binding were both increased in cocaine overdose victims in comparison to age-matched control subjects.

The majority of neurons within the CNS are excitatory glutamatergic neurons, which often innervate and are in turn modulated by the activity of slower conducting monoaminergic brainstem neurons.

Evidence for dopaminergic involvement in psychosis stems from the observation by others that the clinical efficacy of drugs alleviating psychotic symptoms is proportional to the D2 dopamine receptor antagonist affinity, and that compounds releasing DA such as cocaine and amphetamine are psychotomimetic. The evidence for glutamatergic involvement in psychosis stems from the psychotomimetic properties of NMDA glutatmate receptor antagonsists such as PCP and ketamine. Glutamatergic and dopaminergic systems interact through corticofugal excitatory glutamatergic projections from PFC and amygdala to VTA and NA. The VTA, in turn, sends dopaminergic projections to NA, PFC, and amygdala.

The relative affinities of dopamine receptors and distribution of the D3 receptor in human brain suggest that the hypothesized homeostatic response through the D3 signaling pathways occur. It is believed in the present invention that this may lead to sensitization in limbic brain areas, ultimately producing clinical symptoms, including the development of psychosis.

Several clinical studies first suggested that behavioral sensitization is observable in humans. In one study, methamphetamine was administered intravenously to 14 methamphetamine addicts and observed that 12 of them rapidly developed psychosis. Similarly, another study found that patients with a history of methamphetamine-induced psychosis, which had resolved with abstinence, displayed a rapid recurrence of psychosis following even a small exposure to methamphetamine months later. Still further, it has been shown that cocaine-induced psychosis occurred more rapidly after drug ingestion, more frequently, and with the use of less drug over time in patients with cocaine dependence. Although these findings were uncontrolled clinical observations, they are nevertheless consistent with an enduring sensitized response to stimulant ingestion in human subjects.

In the first double-blind, placebo-controlled study by others in healthy volunteers, two doses of d-amphetamine (0.25 mg/kg) alternating with two matched-placebo doses were administered. Amphetamine-induced increases in eye-blink rate and ratings of energy level, mood, and talkativeness were greater following the second d-amphetamine dose as compared to the first d-amphetamine and both placebo doses. In a different sample of eleven healthy volunteers who were administered three single oral doses of d-amphetamine (0.25 mg/kg) alternating with three matched placebo doses in a randomized, double-blind trial, increases in eye-blink rate, elevated mood, talkativeness, and motor activity progressed following each amphetamine dose. Results from these placebo-controlled, double-blind studies suggest that a progression of behavioral responses occurs in healthy volunteers following repeated stimulant administration that is homologous to the progression of behaviors seen in animal models of sensitization.

To examine the clinical relevance of these observations, others have studied the progression of these same behaviors following amphetamine administration in patients with recent-onset psychosis. Carefully selected patients with mild- to moderate-severity psychotic manic and schizophrenic exacerbations and no prior psychotropic medication treatment received two low oral doses of d-amphetamine (0.25 mg/kg) using the same protocol as performed in the healthy volunteers. In contrast to the healthy volunteers, these patients did not demonstrate differences between the two amphetamine doses in any symptoms or behaviors measured. These results suggest that by the time patients developed psychosis or mania, possibly through endogenous enhancement in dopamine neurotransmission, they are fully sensitized so that additional behavioral progression in response to low-dose stimulants cannot occur. In accordance with the present invention, this suggests the presence of a down-regulated (“tolerant”) D3 dopamine system, leading to relatively overactive D2 and D1 dopamine systems that produces the symptoms of psychosis. Consistent therewith, the D3 agonist (+)-PD 128,907 effectively inhibits two behaviors utilized as preclinical models of antipsychotic drug efficacy, stereotyped behaviors induced by apomorphine and dizocilipine. The efficacy of (+)-PD 128,907 in this behavioral assay was stereospecific and blocked by the selective D3 receptor antagonist NGB 2900, suggesting a potential role for D3 agonists in the treatment of acute psychotic symptoms.

Also consistent with this model of psychosis in which adaptive down-regulation of D3 dopamine receptors contributes to the development of psychotic symptoms, the D3 receptor antagonist (+)-UH232 further worsened positive psychotic symptoms in schizophrenia patients following a single dose. Patients receiving (+)-UH232 in a placebo-controlled study by others showed worsening of symptoms including unusual thought content, anxiety, activation, and hostility during the 8 hours following single dose treatment. These results suggest that psychotic symptoms, similar to rodent locomotion, may be regulated by a balance between D3 and D1/D2 receptor activation. Also consistent therewith, the partial D3 dopamine receptor agonist (−)-3-(3-hydroxyphenyl)-N-n-propylpiperidine [(−)-3PPP] improved psychotic symptoms in schizophrenia patients for up to one week. The therapeutic benefit did not persist with repeated treatment in that study. In a similar fashion, the D3 preferring agonist pramipexole (approximately 7-fold greater relative potency at human D3 relative to human D2 receptor) improved symptoms in 60% of schizophrenia patients when added to treatment with haloperidol in one study.

It is known that an amino acid substitution polymorphism in the amino terminus of the D3 receptor modulates vulnerability to schizophrenia, suggesting an interaction between D3 receptor function and other genetic and environmental factors in mediating development of a chronic psychotic illness. In one study by others decreased D3 dopamine receptor mRNA levels were observed in orbitofrontal cortex of schizophrenia patients in comparison to controls, while another study of schizophrenia subjects observed increased D3 receptor binding in schizophrenia subjects who had not received antipsychotic medication in the month prior to death.

Accordingly, we believe that an imbalance in homeostatic responses between D3 and D1/D2 signaling pathways to increased dopamine preceding the onset of psychosis is a critical, common determinant contributing to the development of psychosis. Since homeostatic responses to increased D3 receptor occupation will occur in all systems interfacing with D3 pathways, this suggests a dysfunction in any of these multiple systems plays a role in vulnerability to psychosis. On this basis, in accordance with the present invention, a D3 antagonist is administered to individuals which are non-symptomatic for psychosis, but are determined by family history or genetic marker or environmental factors to be at high-risk for developing psychoses, or is administered to individuals in the prodromal phases of psychosis, such as schizophrenia and bipolar affective disorder to prevent the occurrence of the symptoms of psychosis.

A number of D3 antagonists are available which are suitable for use in the present invention. Most preferred is nafadotride and certain naphthamide derivatives which act selectively as D3 antagonists. A list of suitable naphthamide derivatives which are preferred for use in the present invention are those set forth in U.S. Pat. No. 5,498,628 entitled, Naphthamide Derivatives, the entire disclosure of which is incorporated herein by reference. Additional D3 antagonists which may be suitable or preferred in use in the present invention are: NGB 2849, U99194A, (+)-AJ 76, (+)-UH 232, PD 152255, NGB 2904, S33084, GR 103,691, GR 218,231, (+)-S 14297, or SB-277011.

The evaluation of the patient for factors associated with the risk of developing psychosis preferably includes determining whether the following prodromal symptoms exist: a reduction in the ability to concentrate; a reduction in personal motivation; depressed mood; sleep disturbances; anxiety; social withdrawal; suspiciousness; deterioration in role functioning; and irritability. In addition, or in the alternative, non-symptomatic factors such as a family history of psychosis or biological markers, or environmental or other risk factors such as current presence of the subject (patient) in an intensive care unit for treatment of a medical or surgical illness, indicative of a high risk of developing psychosis are assessed.

After reaching a diagnosis that the patient is either at high risk for developing psychosis or has entered the prodromal phase of psychosis, a selective D3 antagonist is administered. The precise dose and dosage regimen for use in the present invention is a function of variables such as the subject's age, weight, medical history and the like as well as the characteristics of the selective D3 antagonist administered. In general, the preferred dose and dosage regimen based on the weight of the D3 antagonist (i.e., disregarding carrier) effective in the suppression of psychotic symptoms is a daily dose of about 1 ug to about 10 mg per kg of body weight of the patient. The D3 antagonists are administered orally or parenterally. As used herein, “preventing the symptoms” shall mean a reduction in manifestation of symptoms of psychosis in number or severity.

As used herein “psychosis” shall include, but is not limited to, psychosis which occurs as part of conditions including schizophrenia, bipolar disorder, schizoaffective disorder, delirium, dementia and schizophreniform disorder.

We claim:

1. A method of treating a non-psychotic human subject to prevent the development of psychotic symptoms, comprising the steps of:
evaluating the subject to determine the presence of pre-psychotic prodromal;
making a diagnosis that the subject has a high-risk of developing psychosis; and
administering a selective D3 antagonist in an amount and dosage regimen sufficient to suppress psychotic symptoms in said subject.

2. The method of treating a non-psychotic human subject to suppress the development of psychotic symptoms recited in claim 1, wherein the D3 antagonist is selected from the group consisting of nafadotride, naphthamide derivatives which act selectively as D3 antagonists, NGB 2849, U99194A, (+)-AJ 76, (+)-UH 232, PD 152255, NGB 2904, S33084, GR 103,691, GR 218,231, (+)-S 14297, or SB-277011.

3. The method of treating a non-psychotic human subject to suppress the development of psychotic symptoms recited in claim 1, wherein the amount of D3 antagonist administered is from about 1 ug/kg to about 10 mg/kg of subject body weight in a 24 hour period.

4. The method of treating a non-psychotic human subject to prevent the development of psychotic symptoms recited in claim 1, wherein said D3 antagonist is administered either orally or parenterally.

5. The method of treating a non-psychotic human subject to prevent the development of psychotic symptoms recited in claim 1, wherein said pre-psychotic prodromal symptoms include a reduction in the ability to concentrate; a reduction in personal motivation; depressed mood; sleep disturbances; anxiety; social withdrawal; suspiciousness; deterioration in role functioning; and irritability.

6. The method of treating a non-psychotic human subject to prevent the development of psychotic symptoms in claim 1, wherein said diagnosis further includes consideration of at least one prodromal risk factor

7. The method of treating a non-psychotic human subject to suppress the development of psychotic symptoms recited in claim 6, wherein said non-symptomatic risk factor is family history of psychosis.

8. A method of treating a non-psychotic human subject to suppress the development of psychotic symptoms, comprising the steps of:
evaluating a subject for non-symptomatic risk factors indicative of a predisposition for developing psychosis;
making a diagnosis that the subject is at high risk for developing psychosis; and
administering a selective D3 antagonist in an amount and dosage regimen sufficient to suppress psychotic symptoms in said subject.

9. The method of treating a non-psychotic human subject to prevent the development of psychotic symptoms recited in claim 8, wherein the D3 antagonist is selected from the group consisting of nafadotride, naphthamide derivatives which act selectively as D3 antagonists, NGB 2849, U99194A, (+)-AJ 76, (+)-UH 232, PD 152255, NGB 2904, S33084, GR 103,691, GR 218,231, (+)-S 14297, or SB-277011.

10. The method of treating a non-psychotic human subject to prevent the development of psychotic symptoms recited in claim 8, wherein the amount of D3 antagonist administered is from about 1 ug/kg to about 10 mg/kg of subject body weight in a 24 hour period.

11. The method of treating a non-psychotic human subject to prevent the development of psychotic symptoms recited in claim 8, wherein said D3 antagonist is administered either orally or parenterally.

12. The method of treating a non-psychotic human subject to suppress the development of psychotic symptoms recited in claim 8, wherein said non-symptomatic risk factor is family history of psychosis.

13. The method of treating a non-pscyhotic human subject to prevent the development of psychotic symptoms recited in claim 8, wherein said risk factors include environmental factors.

14. The method of treating a non-pscyhotic human subject to prevent the development of psychotic symptoms recited in claim 8, wherein said environmental factors include current presence in an intensive care unit.
